(19) United States
(12) Patent Application Publication
(10) Pub. No.: US 2024/0148971 A1
(43) Pub. Date: May 9, 2024

(54) PROGRAMMATIC MEDICAMENT TITRATION WITH A MEDICAMENT DELIVERY DEVICE

(71) Applicant: INSULET CORPORATION, Acton, MA (US)

(72) Inventors: Matthew ALLES, Winchester, MA (US); Yibin ZHENG, Hartland, WI (US); Joon Bok LEE, Acton, MA (US); James CAUSEY, Simi Valley, CA (US); Jason O'CONNOR, Acton, MA (US)

(21) Appl. No.: 18/386,647

(22) Filed: Nov. 3, 2023

Related U.S. Application Data
(60) Provisional application No. 63/434,238, filed on Dec. 21, 2022.
(60) Provisional application No. 63/382,177, filed on Nov. 3, 2022.

(51) Int. Cl. A61M 5/172; A61M 5/142

(52) U.S. Cl. CPC A61M 5/1723; A61M 5/14248; A61M 2202/0486

(57) ABSTRACT

Exemplary embodiments may provide an on-body medicament delivery system that provides basal delivery of a medicament to a type 2 diabetes patient and that automatically performs medicament titration for the patient. The medicament delivery system performs medicament titration based on glucose level readings for the patient. These glucose levels may be provided wirelessly from a glucose sensor, such as a continuous glucose monitor, or may be entered manually by the patient into a management device, such as a smartphone running an application that provides a user interface for the patient to enter the glucose level readings. The medicament delivery system adjusts the basal medicament delivery rate/dose based on the glucose level readings for the patient. The adjustments may be performed by a programmatic mechanism, such as by computer programming instructions executing on a processor.

### RELATED APPLICATIONS

This application claims the benefit of U.S. Provisional Patent Application No. 63/382,177, filed Nov. 3, 2022, and U.S. Provisional Patent Application No. 63/434,238, filed Dec. 21, 2022, the entire contents of which are incorporated herein by reference in their entirety.

### BACKGROUND

Insulin titration for type 2 diabetes patients seeks to determine a suitable daily delivery rate for delivering insulin to the patient. Conventionally, insulin titration for a type 2 diabetes patient can be an onerous task. Typically, a healthcare provider (HCP) prescribes and directs the patient to take a single daily injection of long-acting insulin at an initial dose of 10 units of insulin or at another dose based on the weight of the patient as determined by American Diabetes Association (ADA) guidelines. The HCP may then give the patient an insulin pen for self-delivery of insulin for on-going treatment of type 2 diabetes. The HCP provides instructions to increase the daily insulin injection every few days by a specified amount. This process continues until a daily dosage is settled upon that will enable the patient to maintain a stable glucose level. The titration period can vary from a few months to years, based on the progression of the patient's condition.

Insulin titration is complicated by the need to vary insulin delivery levels to the patient due to diet, exercise and other activities that affect the glucose level of the patient. The patient may, in some instances, reduce a daily insulin amount to reduce the risk of a hypoglycemic event. Conversely, the patient may need to increase daily insulin delivery levels due to the patient's diet, in order to avoid a hyperglycemic event.

### SUMMARY

In accordance with an inventive facet, a medicament delivery device is configured for delivering basal or background medicament doses to a type two diabetes patient. The device includes a non-transitory computer-readable storage medium storing computer programming instructions and a processor configured for executing the computer programming instructions. Executing the computer programming instructions causes the processor to establish an initial basal or background medicament delivery rate for delivery of medicament to the diabetes patient and to receive glucose level readings of the patient over an initial time window since establishing the initial medicament delivery rate. Executing the computer programming instructions also causes the processor to adjust the medicament delivery rate to a new delivery rate for the diabetes patient based on the received glucose level readings as part of the titration, and the processor causes the delivery of medicament to the diabetes patient at the new delivery rate for the next time window. The medicament may be, for example, insulin, a glucagon-like peptide-1 (GLP-1) receptor agonist, a glucose-dependent insulinotropic polypeptide (GIP) receptor agonist, a dual GLP-1 and GIP receptor agonist, other type of antihyperglycemic medicament, or a co-formulation thereof.

The processor may be further configured to receive input and establish the initial basal medicament delivery rate based at least in part on the received input. The input may include at least one of an age of the patient, a weight of the patient, a gender of the patient, a starting medicament dose, or a titration frequency. The input may include a titration frequency, and the titration frequency may determine a length of the initial time window and the next window. The input may be received wirelessly from a management device that manages the medicament delivery device. The management device may be a smartphone in some embodiments. The processor may be further configured to receive a request to pause medicament delivery and to pause delivery of medicament in response to receipt of the request. The processor may be further configured to receive an indication that a patient will be in a state that will require less medicament during a portion of the next time window and is further configured to reduce the new medicament delivery rate during the portion. The processor may be further configured to detect that the glucose level for the patient is below a threshold and to suspend delivery of medicament in response to the detecting. The processor may be further configured to receive input indicating that the patient is about to sleep or about to begin an activity that will lower their glucose level and to adjust the new delivery rate to a lower delivery rate to compensate. The processor may be further configured to adjust the new delivery rate based on time of day. The processor may be further configured to remove an impact of meal consumption on the received glucose level readings in setting the new delivery rate.

In accordance with a further inventive facet, a management device for an medicament delivery device includes a non-transitory computer-readable storage medium storing computer programming instructions and a display. The management device further includes a processor that is configured for executing the computer programming instructions. Executing the computer programming instructions causes the processor to display on the display a user interface to obtain input regarding gender, age, and/or weight of the patient, and to obtain the input regarding gender, age, and/or weight of the patient. Executing the computer programming instructions additionally causes the processor to forward the input regarding gender, age, and/or weight of the patient to the medicament delivery device for the user to initiate titration of an medicament delivery rate to the patient. The medicament may be, for example insulin, a glucagon-like peptide-1 (GLP-1) receptor agonist, a glucose-dependent insulinotropic polypeptide (GIP) receptor agonist, a dual GLP-1 and GIP receptor agonist, an antihyperglycemic medicament, or a co-formulation thereof.

The obtained input also may include an identification of a starting medicament dose at which the titration is to begin and an indication of how often to titrate. The processor may be further configured to display another user interface to receive an indication that the patient is about to sleep or is about to exercise and to forward the received indication to the medicament delivery device.

In accordance with an additional inventive facet, an medicament delivery device is configured for delivering basal medicament doses to a diabetes patient. The device includes a non-transitory computer-readable storage medium storing computer programming instructions and a processor configured for executing the computer programming instructions. Executing the computer programming instructions causes the processor to establish an initial basal delivery rate of medicament for delivery to the patient by the medicament delivery device over an initial time window. Executing the computer programming instructions may further cause the processor to increase the basal delivery rate in subsequent time windows based on glucose level readings for the patient and may decrease the basal delivery rate to a decreased basal rate for a time window that may be after the time windows where the basal delivery rate is increased. The basal delivery rate in subsequent time windows may continue to be increased or decreased in a similar fashion to maintain euglycemia. Where the glucose level readings of the patient for the later time window exhibit good glucose level control, the titration is concluded by setting the decreased basal rate as the basal delivery rate for the patient. The medicament may be, for example, insulin, a glucagon-like peptide-1 (GLP-1) receptor agonist, a glucose-dependent insulinotropic polypeptide (GIP) receptor agonist, a dual GLP-1 and GIP receptor agonist, an antihyperglycemic, or a co-formulation thereof.

Executing the computer programming instructions may cause the processor to calculate average glucose levels for glucose readings of the time windows. Executing the computer programming instructions may cause the processor, for the time windows, to calculate differences between the average glucose levels of the time windows and target glucose levels. The increases or decreases of the basal delivery rate are based on the calculated differences. The device may be an insulin pump.

### BRIEF DESCRIPTION OF THE DRAWINGS

FIG. 1 depicts an illustrative medicament delivery system that is suitable for exemplary embodiments.

FIG. 2 depicts components of the medicament delivery system of exemplary embodiments when operating in automatic mode.

FIG. 3 depicts an illustrative plot of glucose level readings and basal delivery rates over time when operating in automatic mode.

FIG. 4 depicts illustrative user interfaces of the management device in exemplary embodiments for downloading an application on the management device and gathering patient information as part of first time setup for a first time medicament delivery device operating in automatic mode.

FIG. 5 depicts illustrative user interfaces of the management device in exemplary embodiments for filling a reservoir of the medicament delivery device with medicament and deploying the cannula as part of the first time setup in automatic mode.

FIG. 6 depicts illustrative user interfaces of the management device relating to the cannula deployment and providing information regarding the medicament delivery device in automatic mode in exemplary embodiments.

FIG. 7 depicts illustrative user interfaces of the management device in exemplary embodiments that assist the patient in activating a continuous glucose monitor (CGM) and establishing wireless communication with the CGM in automatic mode.

FIG. 8 depicts illustrative user interfaces of the management device in exemplary embodiments relating to proposed basal delivery adjustments when operating in automatic mode.

FIG. 9 depicts illustrative user interfaces of the management device in exemplary embodiments for displaying glucose level and medicament delivery trends when in automatic mode.

FIG. 10 depicts illustrative user interfaces of the management device in exemplary embodiments for displaying a basal delivery rate trend when in automatic mode.

FIG. 11 depicts components of the medicament delivery system of exemplary embodiments when operating in manual mode.

FIG. 12 depicts an illustrative plot of blood glucose readings and basal delivery rates over time when operating in manual mode.

FIG. 13 depicts an illustrative user interface of the management device in exemplary embodiments providing information regarding the medicament delivery device in manual mode.

FIG. 14 depicts illustrative user interfaces of the management device for manually entering blood glucose level readings of the patient in exemplary embodiments.

FIG. 15 depicts illustrative user interfaces of the management device in exemplary embodiments relating to prompting the patient to update the glucose level reading in manual mode and displaying the updated glucose level reading.

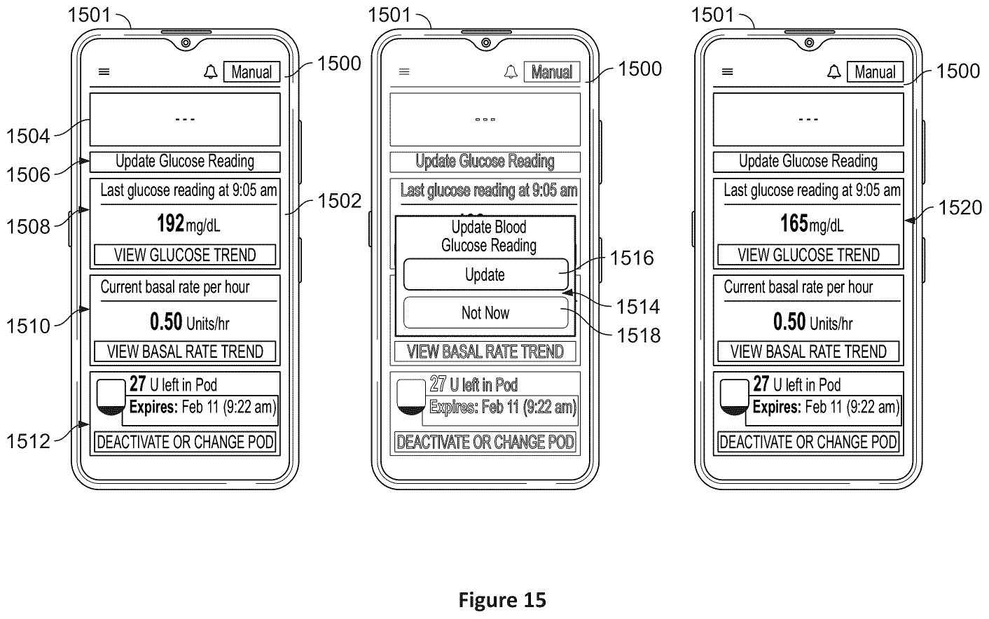

FIG. 16 depicts illustrative user interfaces of the management device for requesting acceptance of an adjusted basal delivery rate and displaying the updated basal delivery rate after acceptance in exemplary embodiments.

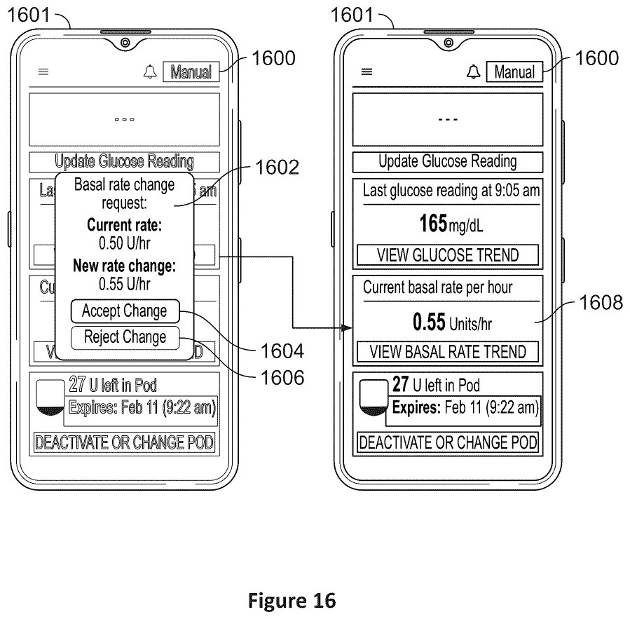

FIG. 17 depicts illustrative user interfaces of the management device in exemplary embodiments for displaying blood glucose level trends when in manual mode.

FIG. 18 depicts illustrative user interfaces of the management device in exemplary embodiments for displaying a basal delivery rate trend when in manual mode.

FIG. 19 depicts a flowchart of illustrative steps that may be performed in exemplary embodiments in the titration process.

FIG. 20 depicts a flowchart of illustrative steps that may be performed in exemplary embodiments where the trigger is the elapsing of a time window.

FIG. 21 depicts a flowchart of illustrative steps that may be performed in exemplary embodiments in performing the titration adjustment process.

FIG. 22 depicts a flowchart of illustrative steps that may be performed in exemplary embodiments to adjust the basal delivery rate as part of the titration.

FIG. 23 depicts a flowchart of illustrative steps that may be taken in exemplary embodiments to use different step sizes.

FIG. 24 depicts a flowchart of illustrative steps that may be performed in exemplary embodiments to suspend basal delivery in some circumstances.

FIG. 25 depicts a flowchart of illustrative steps that may be performed in exemplary embodiments to adjust the basal delivery rate when the glucose level of a patient falls below a hypoglycemic threshold.

FIG. 26 depicts a flowchart of illustrative steps that may be performed in exemplary embodiments to adjust the basal delivery rate based on time of day.

FIG. 27 depicts a flowchart of illustrative steps that may be performed in exemplary embodiments to account for elevated glucose levels.

FIG. 28 depicts a flowchart of illustrative steps that may be performed in exemplary embodiments to remove post-prandial glucose level readings identified by the patient when calculating the average glucose level for a time period.

FIG. 29 depicts a flowchart of illustrative steps that may be performed in exemplary embodiments to detect whether the user is sleeping and to respond to detecting that the user is sleeping.

FIG. 30 depicts a flowchart of illustrative steps that may be performed in exemplary embodiments to detect whether the user is exercising and to respond to detecting that the user is exercising.

FIG. 31 depicts a flowchart of illustrative steps that may be performed by exemplary embodiments to switch from a collocated glucose sensor to an external glucose sensor.

FIG. 32 depicts a flowchart of illustrative steps that may be performed in exemplary embodiments with a reduced accuracy glucose monitor.

FIG. 33 depicts a flowchart of illustrative steps that may be performed in exemplary embodiments to address instances when such a reduced accuracy glucose monitor becomes interrupted for a period.

FIG. 34 depicts a flowchart of illustrative steps that may be performed in exemplary embodiments to adjust the total daily insulin (TDI) for a user based on activity level.

FIG. 35 depicts a flowchart of illustrative steps that may be performed in exemplary embodiments to adjust the TDI for the user based on activity level per a first approach.

FIG. 36 depicts a flowchart of illustrative steps that may be performed in exemplary embodiments to adjust the TDI for the user based on activity level per a second approach.

### DETAILED DESCRIPTION

Exemplary embodiments may provide an on-body medicament delivery system that provides basal or background delivery of medicament to a diabetes patient and that automatically performs medicament titration for the patient. The medicament may be, for example, insulin, a glucagon-like peptide-1 (GLP-1) receptor agonist, a glucose-dependent insulinotropic polypeptide (GIP) receptor agonist, a dual GLP-1 and GIP receptor agonist, an antihyperglycemic medicament, or a co-formulation thereof. Examples are discussed below where the medicament is insulin. These examples are intended to be illustrative and not limiting. The medicament delivery system performs medicament titration based on glucose level readings for the patient. These glucose level readings may be provided wirelessly from a glucose sensor, such as a continuous glucose monitor, or may be entered manually by the patient into a management device, such as a smartphone running an application that provides a user interface for the patient to enter the glucose level readings. The medicament delivery system adjusts the basal medicament delivery rate/dose based on the glucose level readings for the patient. The adjustments may be performed by a programmatic mechanism, such as by computer programming instructions executing on a processor. The exemplary embodiments may quickly and accurately perform the medicament titration without the need for the primary care physician or HCP to constantly monitor the patient and make frequent decisions as to how to adjust the medicament delivery rate/dose. Moreover, the patient need not try to determine how to adjust medicament delivery amounts to account for factors like diet and exercise that may vary from day to day. The programmatic mechanism may account for such factors.

The exemplary embodiments may enable the patient or HCP to specify a time window in which the patient typically sleeps. The medicament delivery device, in response, may adjust the medicament delivery rate/dose for the time period where the patient sleeps. The exemplary embodiments may enable a patient to specify that the patient is to perform an activity, such as exercise, during a time window. The exemplary embodiments may adjust the medicament delivery rate/dose for the time window to account for the effect the activity is anticipated to have on the glucose level of the patient. Other adjustments may be made for time of day, experiencing a hypoglycemic event, a hyperglycemic event, or the like.

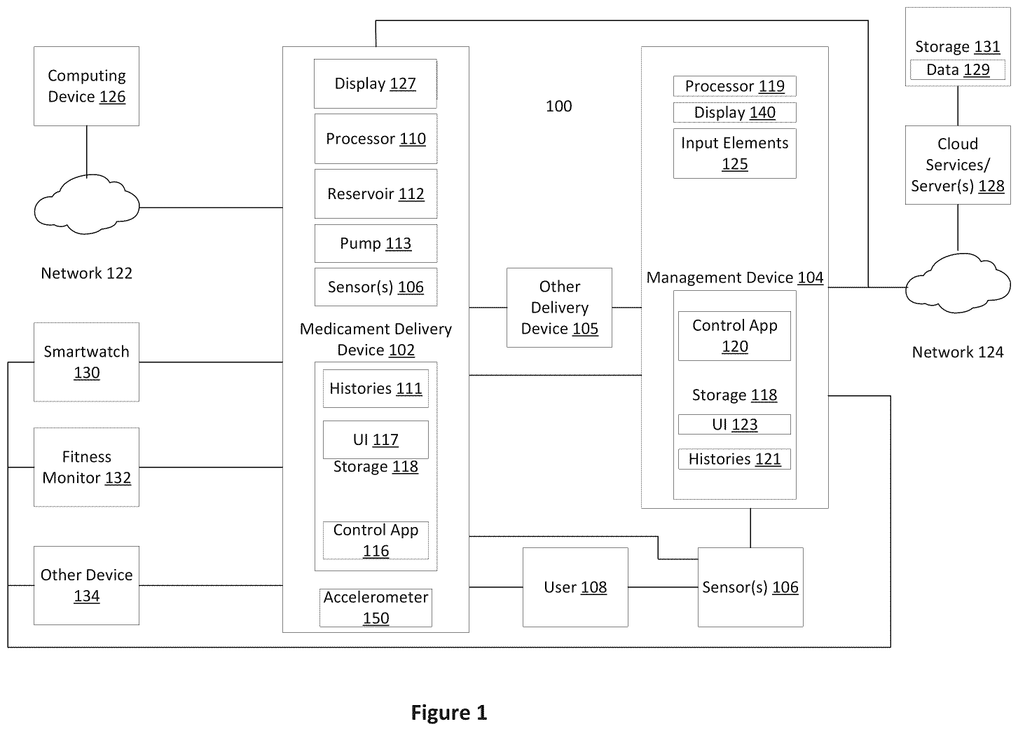

FIG. 1 depicts a block diagram of an illustrative medicament delivery system 100 that is suitable for delivering medicament to a user 108 (e.g., a type 2 diabetes patient) in accordance with the exemplary embodiments. As mentioned above, the medicament may be, for example, insulin, a glucagon-like peptide-1 (GLP-1) receptor agonist, a glucose-dependent insulinotropic polypeptide (GIP) receptor agonist, a dual GLP-1 and GIP receptor agonist, an antihyperglycemic, or a co-formulation thereof. The medicament delivery system 100 includes a medicament delivery device 102. The medicament delivery device 102 may be a wearable device that is worn on the body of the user 108 or carried by the user. The medicament delivery device 102 may be directly coupled to a user (e.g., directly attached to a body part and/or skin of the user 108 via an adhesive or the like) with no tubes and an infusion location directly under the medicament delivery device 102, or may be carried by the user (e.g., on a belt or in a pocket) with the medicament delivery device 102 connected to an infusion site where the medicament is injected using a needle and/or cannula. A surface of the medicament delivery device 102 may include an adhesive to facilitate attachment to the user 108.

The medicament delivery device 102 may include a processor 110. The processor 110 may be, for example, a microprocessor, a logic circuit, a field programmable gate array (FPGA), an application specific integrated circuit (ASIC) or a microcontroller. The processor 110 may maintain a date and time as well as other functions (e.g., calculations or the like). The processor 110 may be operable to execute a control application 116 encoded in computer programming instructions stored in the storage 114 that enables the processor 110 to direct operation of the medicament delivery device 102. The control application 116 may be a single program, multiple programs, modules, libraries or the like. The processor 110 also may execute computer programming instructions stored in the storage 114 that may include one or more display screens shown on display 127. The display 127 may display information to the user 108 such as user interface 117 and, in some instances, may receive input from the user 108, such as when the display 127 is a touchscreen.

The control application 116 may control delivery of medicament to the user 108 per a control approach like that described herein. The storage 114 may hold histories 111 for the device and/or user, such as a history of basal deliveries, and/or other histories, such as a meal event history, exercise event history, glucose level history, and/or the like. In addition, the processor 110 may be operable to receive data or information. The storage 114 may include both primary memory and secondary memory. The storage 114 may include random access memory (RAM), read only memory (ROM), optical storage, magnetic storage, removable storage media, solid state storage or the like.

The medicament delivery device may include an accelerometer 150, such as a 3-axis accelerometer, for detecting motion and activity by the user 108. The accelerometer 150 may be used to detect whether the user is sleeping, exercising, or performing other activities.

The medicament delivery device 102 may include a tray or cradle and/or one or more housings for housing its various components including a pump 113, a power source (not shown), and a reservoir 112 for storing medicament for delivery to the user 108. A fluid path to the user 108 may be provided, and the medicament delivery device 102 may expel the medicament from the reservoir 112 to deliver the medicament to the user 108 using the pump 113 via the fluid path. The fluid path may, for example, include tubing coupling the medicament delivery device 102 to the user 108 (e.g., tubing coupling a cannula to the reservoir 112), and may include a conduit to a separate infusion site. The medicament delivery device 102 may have operational cycles in which basal doses of medicament are delivered as needed.

There may be one or more elements for enabling communications links with one or more devices physically separated from the medicament delivery device 102 including, for example, a management device 104 of the user and/or a caregiver of the user, sensor(s) 106, a smartwatch 130, a fitness monitor 132 and/or another variety of device 134. The communication links may include any wired or wireless communication links operating according to any known communications protocol or standard, such as Bluetooth®, Wi-Fi, a near-field communication standard, a cellular standard, or any other wireless protocol.

The medicament delivery device 102 may interface with a network 122 via a wired or wireless communications link. The network 122 may include a local area network (LAN), a wide area network (WAN), a cellular network, or a combination thereof. A computing device 126 may be interfaced with the network 122, and the computing device may communicate with the medicament delivery device 102.

The medicament delivery system 100 may include one or more sensor(s) 106 for sensing the levels of one or more analytes. The sensor(s) 106 may be coupled to the user 108 by, for example, adhesive or the like and may provide information or data on one or more medical conditions and/or physical attributes of the user 108. The sensor(s) 106 may be physically separate from the medicament delivery device 102 or may be an integrated component thereof. The sensor(s) 106 may include, for example, glucose monitors, such as CGMs and/or non-invasive glucose monitors. The sensor(s) 106 may include a glucose monitor that is less accurate than a CGM and that uses microneedles but is well-suited for use with the medicament delivery device 102 for treating Type 2 diabetes. Such a glucose sensor may be cheaper than a CGM that is suited for treating Type 1 diabetes. The sensor(s) 106 may include ketone sensors, analyte sensors, heart rate monitors, breathing rate monitors, motion sensors, temperature sensors, perspiration sensors, blood pressure sensors, alcohol sensors, or the like. Some sensors 106 may also detect characteristics of components of the medicament delivery device 102. For instance, the sensors 106 in the medicament delivery device may include voltage sensors, current sensors, temperature sensors and the like.

The medicament delivery system 100 may or may not also include a management device 104. The management device 104 may be a special purpose device, such as a dedicated personal diabetes manager (PDM) device. The management device 104 may be used to program or adjust operation of the medicament delivery device 102. The management device 104 may be any portable electronic device including, for example, a dedicated device, a smartphone, a smartwatch, or a tablet. In the depicted example, the management device 104 may include a processor 119 and a storage 118. The processor 119 may execute processes to manage a user's glucose levels and to control the delivery of the medicament to the user 108. The medicament delivery device 102 may provide data from the sensors 106 and other data to the management device 104. The data may be stored in the storage 118. The processor 119 may also be operable to execute programming code stored in the storage 118. For example, the storage 118 may be operable to store one or more control applications 120 for execution by the processor 119. The control application 120 may be responsible for controlling the medicament delivery device 102. In some exemplary embodiments, the control application 120 provides the adaptability described herein. The storage 118 may store the control application 120, histories 121 like those described above for the medicament delivery device 102, and other data and/or programs.

A display 140, such as a touchscreen, may be provided for displaying information. The display 140 may display user interface (UI) 123. The display 140 also may be used to receive input, such as when it is a touchscreen. The management device 104 may further include input elements 125, such as a keyboard, button, knobs, or the like, for receiving input form the user 108.

The management device 104 may interface with a network 124, such as a LAN or WAN or combination of such networks, via wired or wireless communication links. The management device 104 may communicate over network 124 with one or more servers or cloud services 128. Data, such as sensor values, may be sent, in some embodiments, for storage and processing from the medicament delivery device 102 directly to the cloud services/server(s) 128 or instead from the management device 104 to the cloud services/server(s) 128.

Other devices, like smartwatch 130, fitness monitor 132 and device 134 may be part of the medicament delivery system 100. These devices 130, 132 and 134 may communicate with the medicament delivery device 102 and/or management device 104 to receive information and/or issue commands to the medicament delivery device 102. One or more of the devices 130, 132, and 134 may gather and process data from the accelerometer 150 in some embodiments. Moreover, these devices 13, 132, and 134 may include activity sensors, and may include software that is executed on the devices 130, 132, and 134 to perform tasks as described below. These devices 130, 132 and 134 may execute computer programming instructions to perform some of the control functions otherwise performed by processor 110 or processor 119, such as via control applications 116 and 120. These devices 130, 132 and 134 may include displays for displaying information. The displays may show a user interface for providing input by the patient, such as to request a change or pause in dosage, or to request, initiate, or confirm delivery of a bolus of medicament, or for displaying output, such as a change in dosage (e.g., of a basal delivery amount) as determined by processor 110 or management device 104. These devices 130, 132 and 134 may also have wireless communication connections with the sensor 106 to directly receive analyte measurement data. Another delivery device 105, such as an medicament delivery pen, may be accounted for or may be provided for also delivering medicament to the user 108.

The functionality described herein for the exemplary embodiments may be under the control of or performed by the control application 116 of the medicament delivery device 102 or the control application 120 of the management device 104. In some embodiments, the functionality wholly or partially may be under the control of or performed by the cloud services/servers 128, the computing device 126 or by the other enumerated devices, including smartwatch 130, fitness monitor 132 or another device 13 that may be wearable.

In the closed loop or automatic/automated mode, the control application 116, 120 determines the medicament delivery amount for the user 108 on an ongoing basis based on a feedback loop. For an medicament delivery device, the aim of the automated mode is to have the user's glucose level at a target glucose level or within a target glucose range.

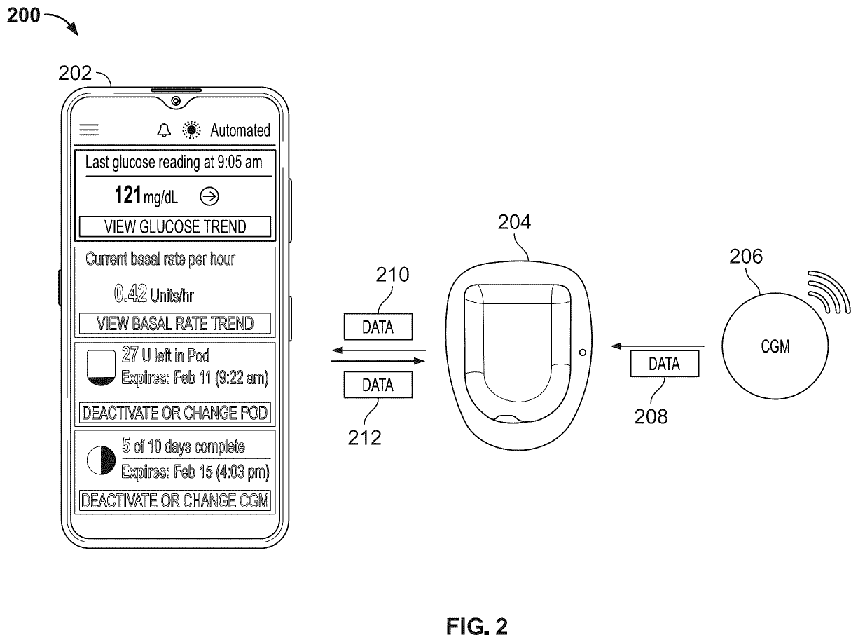

FIG. 2 depicts an illustrative arrangement showing the flow of data between components of an exemplary medicament delivery system 200 when operating in an automatic mode. A smartphone acting as a management device 202 receives data 210 from medicament delivery device 204 and sends data 212 to the medicament delivery device 204 via a wireless communication connection. The medicament delivery device 204 may receive data 208, such as glucose level readings for a patient, from CGM 206. In the automatic mode, the glucose level readings are provided automatically from the CGM 206 to the medicament delivery device 204 at timed intervals. The medicament delivery system 200 may also operate in a manual mode where the user enters glucose level readings, as will be described below. The functionality described below for the management device 202 may be realized by computer programming instructions of application 120 executed by processor 119 of the management device. The functionality described below for the medicament delivery device 204 may be performed by computer programming instructions of the control application 116. The management device application 120 may provide user interface 123, which may include elements as described below and may include elements for receiving input for the medicament delivery device. The medicament delivery device 102 may use information provided by the management device 104 to perform the titration described below and the associated control functions.

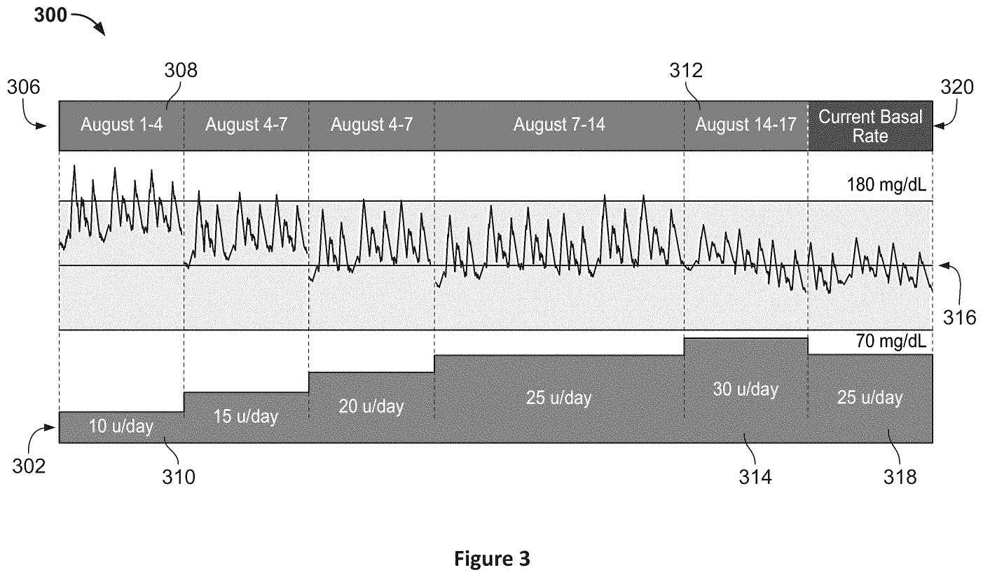

FIG. 3 depicts an illustrative plot 300 of the delivery rate of a medicament, insulin, 302 and the glucose level 304 of the patient for time increments 306 during a titration process with the medicament delivery device 102 operating in automatic mode. As can be seen, in the initial time frame 308 of August 1-4, the glucose level of the patient is high, and the basal delivery rate 310 may be 10 units per day, which may be set by either the patient or HCP. The basal delivery rate may be increased in 5 unit steps if the user's blood glucose readings are not yet in a desired range, so that by the time frame 312 of August 14-17, the basal delivery rate 314 is up to 30 units per day, in this example. This amount may be excessive, as can be seen in this example in the glucose level falling below the target glucose level 316 in the latter portion of the time frame 312. In response, the basal delivery rate 318 may be reduced by 5 unit decrements, for example to 25 units per day for the current time window 320. The glucose level of the patient in this example goes back up closer to the target glucose level 316. Thus, 25 units per day appears to be a good basal delivery rate for the patient. These basal rates and increments/decrements may or may not be a whole number as shown in this example, but rather an effective daily rate for the patient that might vary by time of day. Additionally, since the user is preferably set to start with a low total daily insulin (TDI) level (e.g., 10 units per day in this example), it is often the case that the user's insulin level needs to be titrated upwards (e.g., in increments of 5 units in this example). This may occur in automated fashion (e.g., incrementing and decrementing) until the user's blood glucose levels reach a desired level or range for a period of time, such as a period of one day.

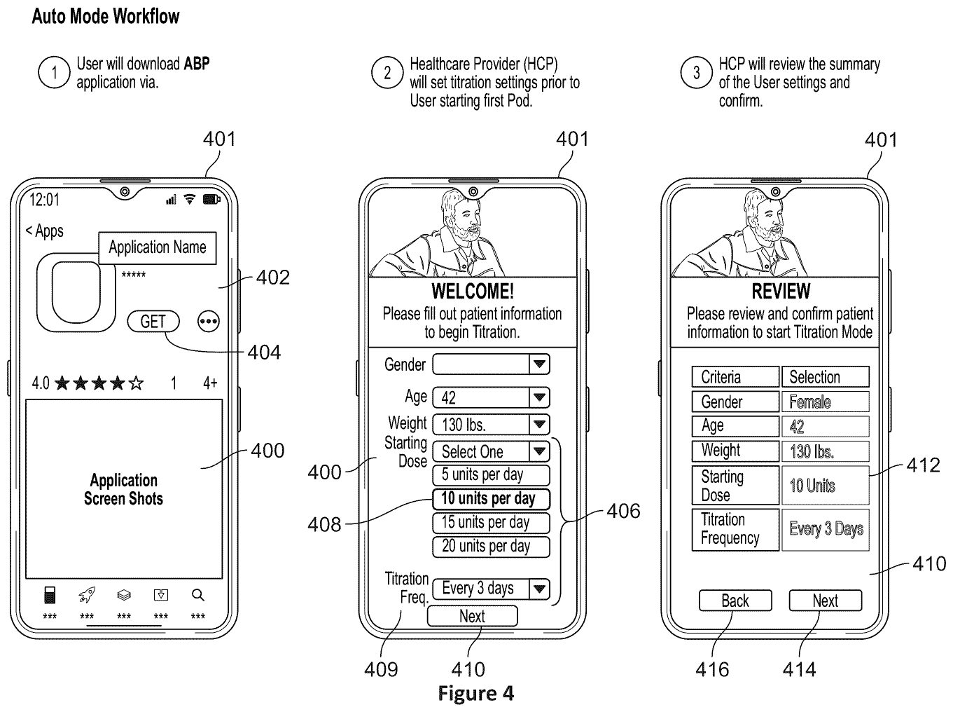

FIG. 4 depicts initial steps that may be performed in exemplary embodiments to configure the medicament delivery device 204 for operation in the automatic mode. In this example, the medicament is insulin, but the process may also apply to titration for user medicaments. Initially, in step 1, the patient may be prompted to download an automatic basal pod (ABP) application, which in some embodiments may be application 120. As can be seen on display 400 of management device 401 (e.g., smartphone), a UI may be shown on display 400 that includes a portion 402 for the ABP application. The portion 402 of the UI identified the application by name and provides a UI element, like button 404, for downloading and installing the ABP application. The patient may activate UI element 404 to cause the downloading and installing.

In step 2, the ABP application has been installed and open. The display 400 of the management device 401 shows a UI that prompts the user or HCP to enter information, such as gender, age, and/or weight. This information may be used in determining a starting basal delivery rate for the patient. For example, the user may enter only their weight (and the application may request solely their weight as an input), and the application may suggest or populate a field for an initial basal delivery rate based on the user's weight. The UI also contains a prompt 406 for specifying the initial basal delivery rate for the patient. The prompt 406 may be a dropdown list of possible basal delivery rates that may be selected by the patient or populated/selected by the application itself based on the user's prior input (e.g., the user's weight). In the example shown, the highlighted rate of 10 units of insulin per day is selected. A UI element 409 prompts the user or HCP to select a titration frequency (i.e., how often to update the basal delivery rate as part of the titration process). Additionally or alternatively, the titration frequency may be a default value, such as every 1 day or every 3 days. Selection of the Next button 410, causes the UI to be updated to show screen 412 on display 400 of the management device as shown in step 3. The UI shows the information entered by the patient or HCP for confirmation or review. Selection of Next button 414 causes the entered information to be accepted. Selection of the Back button 416, return to the previous screen for editing of information edited in step 2.

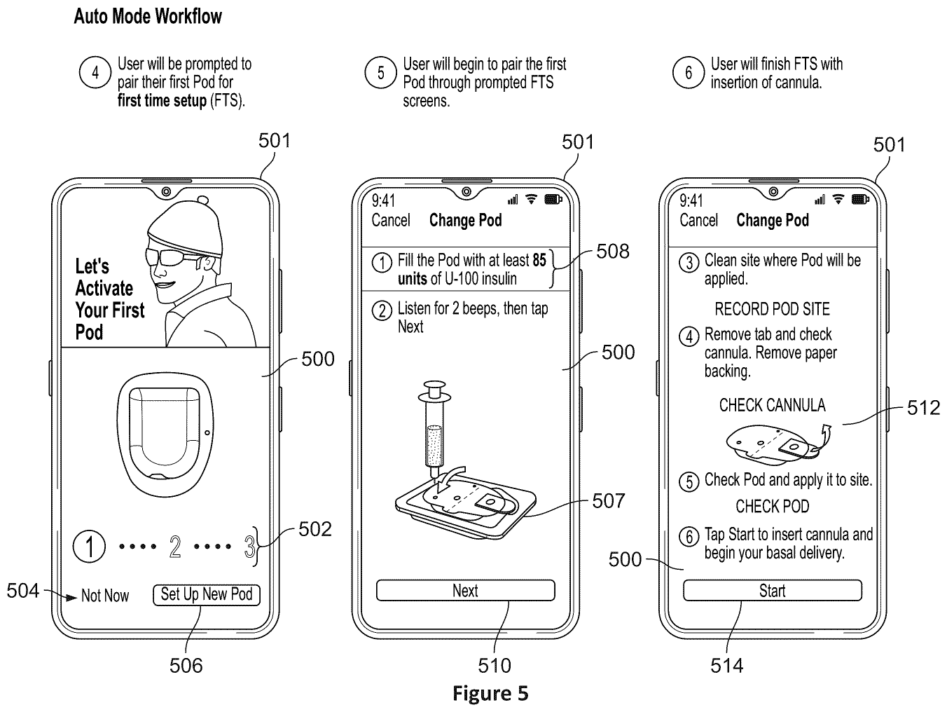

As shown in FIG. 5, in step 4, the patient may be prompted to set up the medicament delivery device 204. The display 500 of the management device 501 may include a status bar 502 for identifying where the patient is in the process of first time setup (FTS) of the medicament delivery device. The patient may select the Not Now button 504 to postpone the pairing or may select the Setup New Pod button 506 to setup the medicament delivery device 204. The display 500 of the management device 501 may then show an image 507 depicting the filling of the reservoir 112 of the medicament delivery device 204 with via a syringe. Text 508 may be displayed that specifies how much medicament (e.g., insulin) should be added to the reservoir 112 based on the current average daily basal rate. In step 6, the display of the management device 501 may be updated to show the instructions for the FTS that ends with insertion of the cannula through the skin of the patient. The patient may select the Start button 514 to cause the cannula insertion.

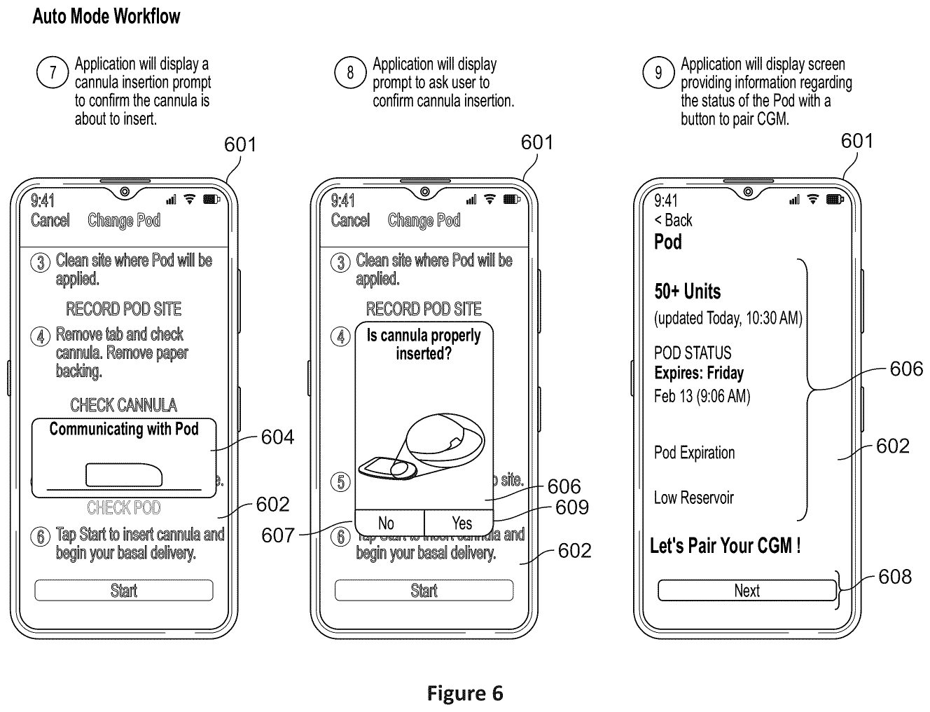

FIG. 6 depicts illustrative steps that may be performed after the Start button is selected by the patient. As shown in step 7, the display 602 of the management device 601 may display a cannula insertion prompt 604 to confirm to the patient that the cannula is about to be inserted. In step 8, the display 602 of the management device 601 may show a prompt 606 to ask the patient to confirm that the cannula insertion has occurred. The patient may select a Yes button 609 to confirm insertion or a No button 607 to indicate no cannula insertion has occurred. In step 9, if the cannula insertion was successful, the display 602 of the management device 601 may show information 606 regarding the medicament delivery device 204, such as how much insulin is in the reservoir 112, and an expiration date and time for the medicament delivery device 204. A Next button 608 may be selected to initiate the pairing with the CGM 206.

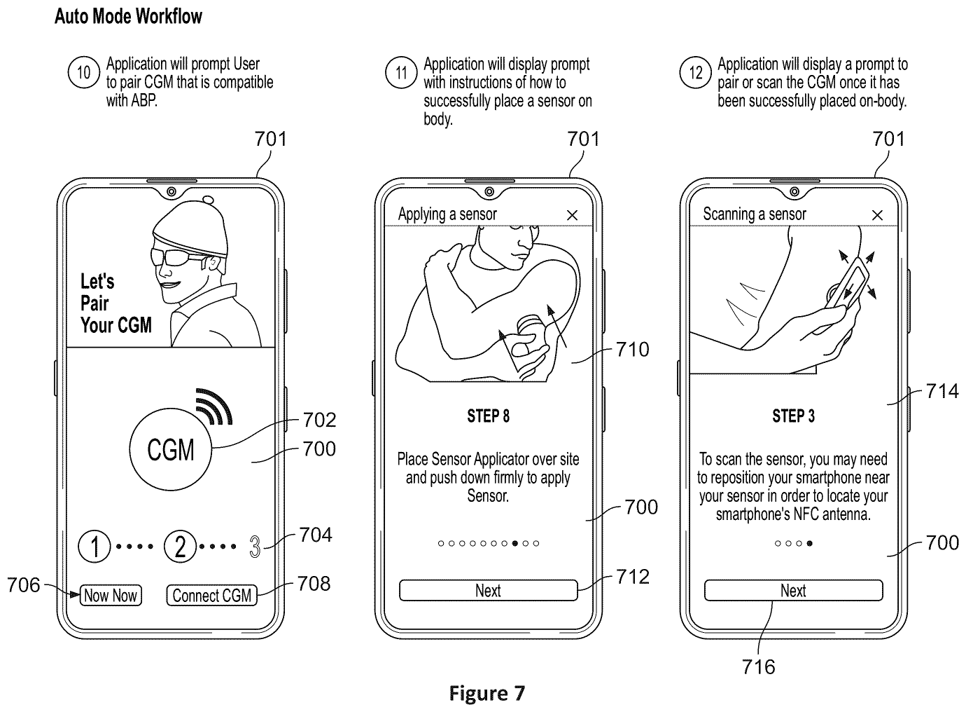

FIG. 7 depicts illustrative initial steps for pairing with the CGM 206. In step 10, the patient may be prompted to take action to pair with the CGM 206. The display 700 of the management device 701, shows an icon indicating the pairing process, and a status bar shows that this is the second step of the FTS. A Not Now button 706 may be selected to halt the process of pairing with the CGM 206. A Connect CGM button 708 may be selected to initiate the pairing with the CGM 206. In step 11, the patient has selected the Connect CGM button 708. The display 700 of the management device 701 may display graphics and text 710 for instructing the patient how to setup and activate the CGM 206. After the patient selects the Next button 712, in step 12, a user interface 714 may be displayed on display 700 of the management device 701 that includes graphics and text to instruct the patient how to initialize or establish communication with the management device 104.

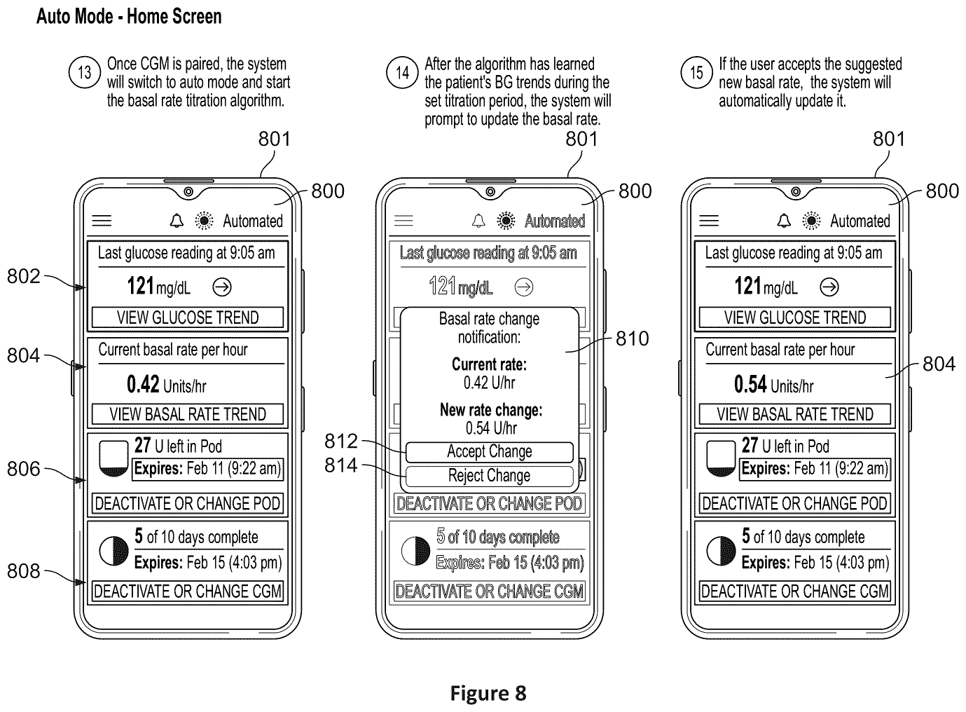

Once the management device 104 is paired with the CGM 206, the medicament delivery system 200 may switch into automatic mode and initiate the medicament titration. FIG. 8 depicts an example of a home screen that may be displayed in step 13. The home screen shown on display 800 of the management device 801 may include a section 802 that displays the most recent blood glucose level reading amount and a trend arrow of the glucose level for the patient. Section 804 of the display 800 may show the current basal delivery rate (e.g., 0.42 units per hour). Section 806 of the display 800 may show an indication of the estimated expiration time and date for the medicament delivery device 204. Section 808 may show how much time there is before the CGM 206 expires.

The medicament delivery device 204 may receive the blood glucose level readings of the patient and may process the received glucose level readings to titrate or adjust the basal delivery rate of medicament by the medicament delivery device 204. At this point, in step 14, the management device 801 may display a prompt 810 on display 800 to prompt the user to review the proposed basal rate change. This proposed basal rate change prompt may be driven by the titration frequency that was set by the HCP or user during FTS. In 810, the user can accept the changed basal delivery rate or not. The patient may select the Accept Change button 812 to accept the change or may select the Reject Change button 814 to reject the change. When prompting the user to accept or reject a new basal delivery rate, the user's amount of time in range (TIR) using the prior basal delivery rate may be displayed to the user to assist the user in determining whether to accept the new suggested basal delivery rate. Additionally or alternatively, an anticipated TIR for the user using the new, suggested basal delivery rate may be shown to the user to assist the user in determining whether to accept the new suggested basal delivery rate, and this anticipated TIR may be based on prior blood glucose values for the user over the previous titration frequency period, and an estimate of the user's insulin-to-carbohydrate ratio, and the new, suggested basal delivery rate, by way of example. As shown in step 15, if the patient accepts the change, the home screen on display 800 of the management device 801 is updated at section 804 to reflect the new basal delivery rate (e.g., 0.54 units per hour) and the basal rate will be updated in the medicament delivery system 200.

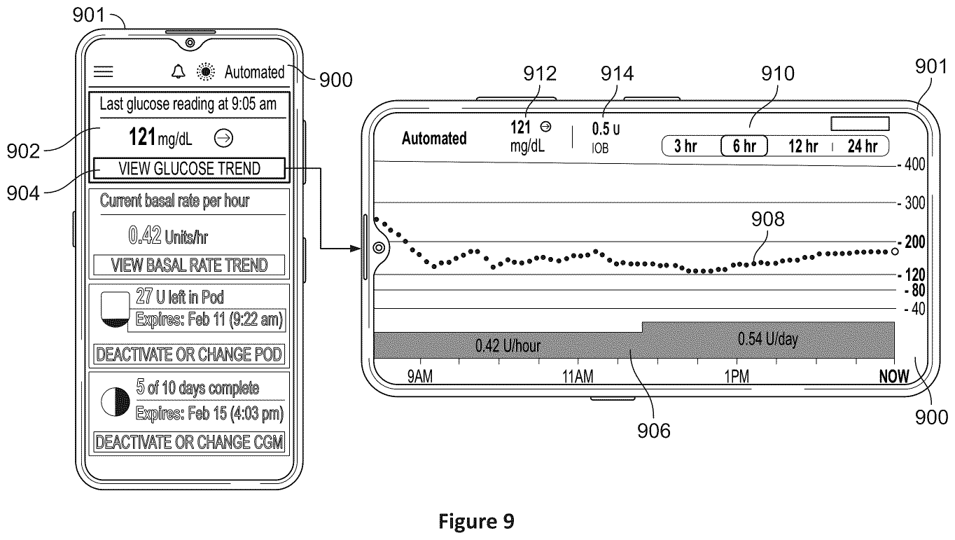

The patient may be able to access additional information from the home screen. For example, as shown in FIG. 9, the home screen shown on display 900 of the management device 901 may include section 902 that displays the current glucose level and a glucose level trend indicator and a View Glucose Trend button 904. Selection of the View Glucose Trend button 904 causes a glucose trend UI to be shown on the display of the management device 801. The glucose trend UI may include a glucose trend curve 908 plotted over time based on the CGM reading frequency and value. The basal delivery rate 906 may be shown over time as well. The current glucose level 912 of the patient, the current basal delivery rate, and/or the current insulin on board 914 may be shown. The patient may select the time frame for the plot using controls 916.

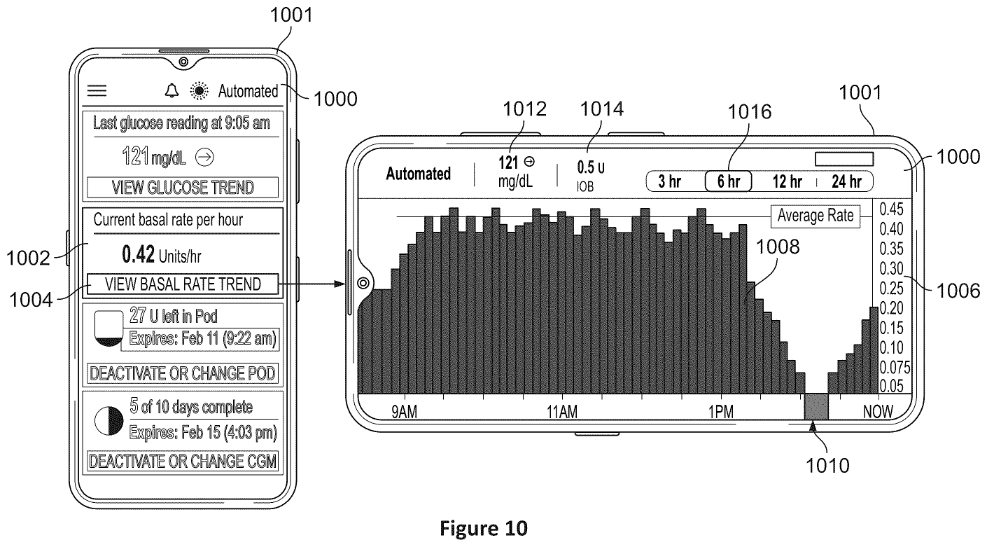

The patient also may be able to navigate to see a plot of the basal delivery rate from the home screen. FIG. 10 shows an illustrative home screen on display 1000 of the management device 1001. Section 1002 of the home screen displays a current basal delivery rate. Section 1002 includes a View Basal Rate Trend button 1004. Selection of the View Basal Rate Trend button 1004 causes a basal delivery rate UI 1006 to be displayed on the display 1000 of the management device 1001. The basal delivery rate UI shows a plot 1008 of the delivery rates over time based on the medicament delivery algorithm frequency of delivery (e.g., every 5 minutes, 10 minutes, or 15 minutes, by way of example). In the example plot, a suspension of basal delivery 1010 is indicated. The current glucose level and trend 1012, the current basal delivery rate, and control for choosing the time frame of the plot may be included as part of the basal delivery rate UI 1006.

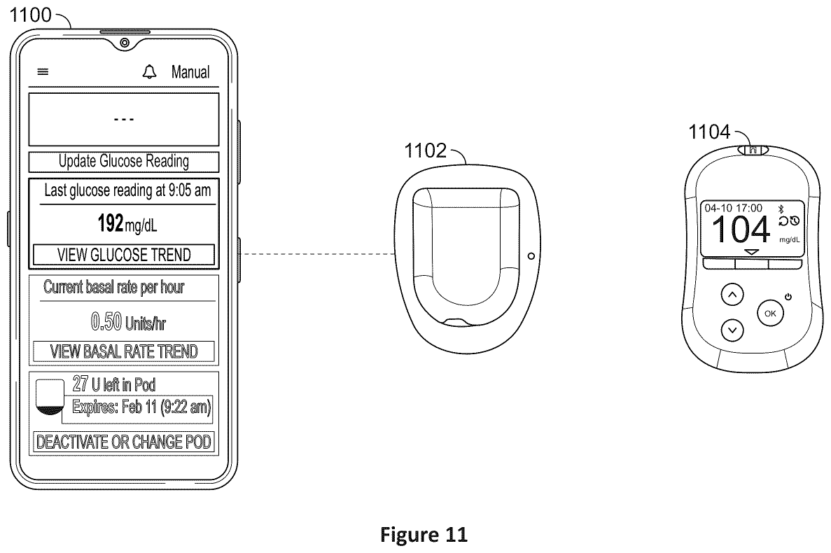

As was mentioned above, the medicament delivery system may also operate in manual mode in which the user manually enters the glucose level readings taken from a blood glucose monitor or continuous glucose monitor that might not be compatible with the system. FIG. 11 depicts an example of several of the key components when operating in the manual mode. The components include a management device 1100 and a medicament delivery device 1102 that may be in wireless communication. The components also may include a standard blood glucose meter 1104 that measure glucose level of the patient from a drop of blood applied to a strip by the patient. There may be wireless connection between the blood glucose meter 1104 and the medicament delivery device 1102 where the blood glucose meter sends a reading via Bluetooth connection when a user takes a blood glucose reading.

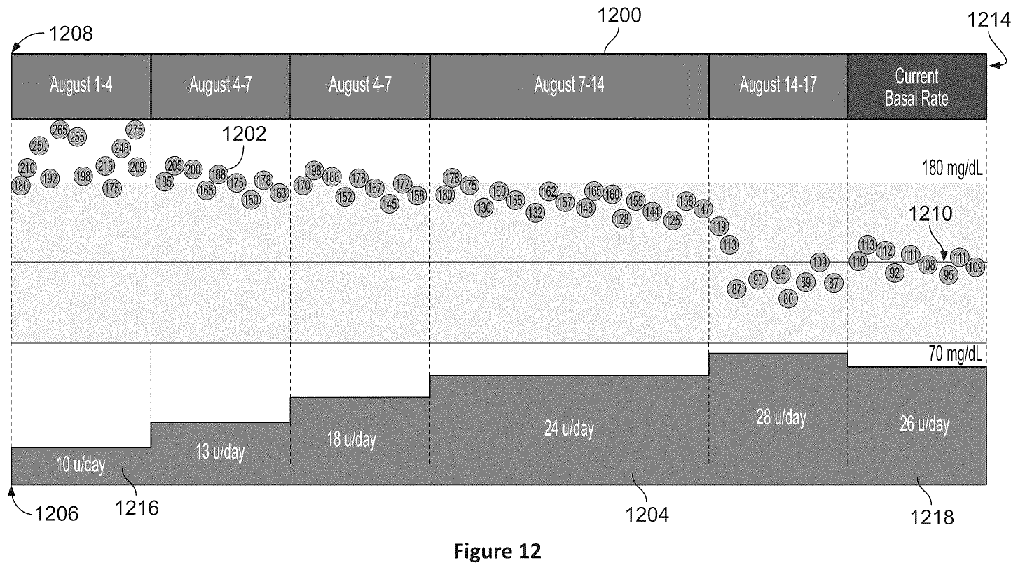

FIG. 12 depicts a plot 1200 of illustrative manually entered glucose level readings 1202 for an instance where the patient is operating the medicament delivery device 1102 in manual mode. The plot 1200 is one of discrete points 1202 over time 1208. The plot 1200 also includes the basal delivery rate for time windows 1206. As can be seen, the basal delivery rate increases until it reaches 28 units per day, and then, in the current time window, is decreased to 26 units per day (see 1218). The plot 1200 also shows the target glucose level 1210 (e.g., 120 mg/dL). As in the case with the automatic mode example of FIG. 3, the glucose level readings of the patient start above the target glucose level 1210 and then decrease as the basal delivery rate increases. When the basal delivery rate reaches 28 units per day, the glucose level readings 1202 are generally below the target glucose level 1210 and need to be increase, so the basal delivery rate is reduced to 26 units per day.

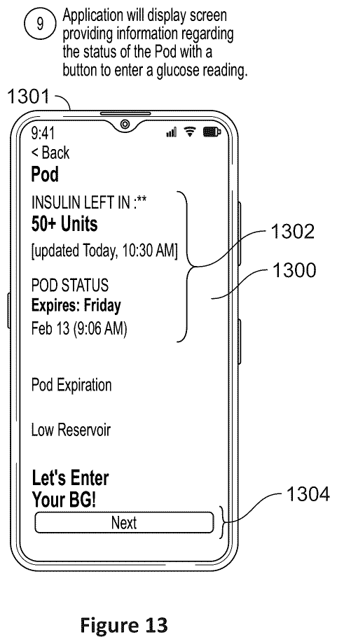

The first time set up process for the medicament delivery device 102 in manual mode is largely the same as for the medicament delivery device 102 in automatic mode through step 8. At step 9 the two processes diverge. As shown in FIG. 13, there is no pairing with the CGM in manual mode. Hence, a user interface is shown on the display 1300 of the management device 1301 that includes information, such as how much medicament has been loaded in the reservoir of the medicament delivery device 102 and the date/time at which the medicament delivery device 102 expires. To start the process of entering manual glucose level readings, the patient selects the Next button 1304.

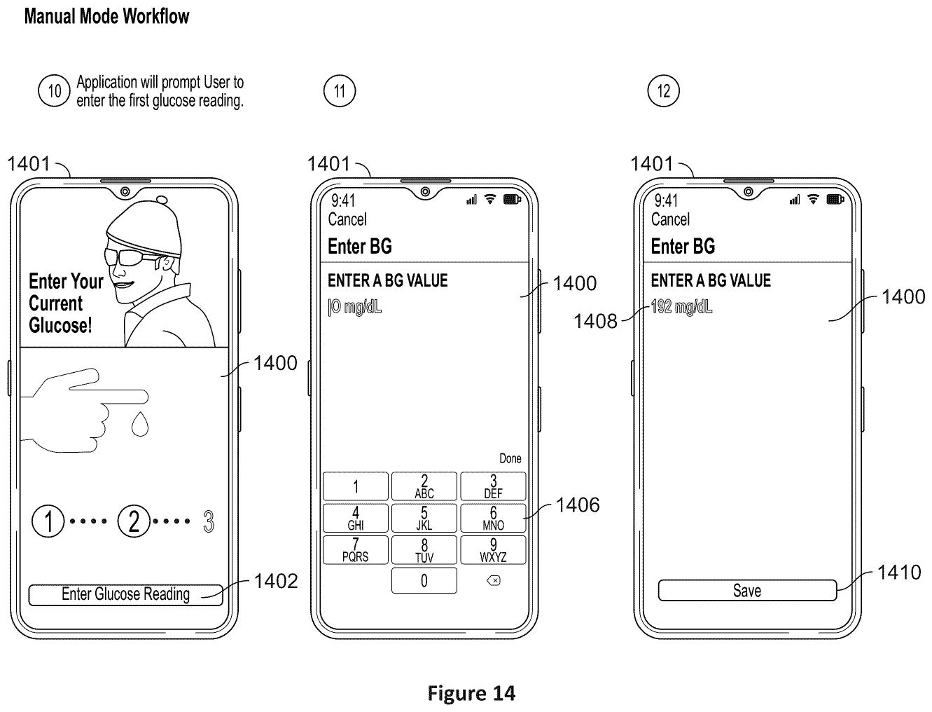

As shown in FIG. 14, when the patient selects the Next button 1304, at step 10, the display 1400 of the management device 1401 shows a UI with an Enter Glucose Reading button 1402. When the patient selects the Enter Glucose Reading Button 1402, at step 11, a UI is displayed on the display that includes a text box for entering a glucose level reading using keypad 1406. Once the glucose level reading has been entered or sent from a paired blood glucose meter, the text box is updated in Step 12, to show the entered value 1408 (e.g., 192 mg/dL). A Save button 1410 may be selected to save the entered glucose level reading.

After the patient enters a most recent glucose level reading, the home screen 1502 may be shown on the display 1500 of the management device 1501. After a period of time, the management device 1501 may prompt the patient to enter an update glucose level reading. The display 1500 may include a section 1504 where the glucose level value is indicated as not being known, such as by showing dashes. Section 1508 shows the last glucose level reading, and section 1510 shows the current basal delivery rate. Section 1512 shows an expiration date and time for the medicament delivery device 1102. A prompt 1514 may pop up at a predetermined time to get the user to enter a new glucose level reading. The prompt may include an Update button 1516 that may be selected to update the glucose level reading and a Not Now button 1518 that may be selected to postpone the updating of the glucose level reading. After the patient enters the new glucose level reading, the glucose level reading 1520 shown may be updated on the display 1500 of the management device 1501. If the system determines too much time has lapsed and cannot predict an accurate basal rate, it may continue the current rate or pause medicament delivery altogether.

The patient may also be prompted to accept an update of the basal delivery rate while viewing the home screen. Specifically, the management device 1601 may show a prompt 1602 on the display 1600 of the management device 1601. The prompt 1602 may identify the current basal delivery rate and the proposed new basal delivery rate. The prompt may include an Accept Change button 1604 that may be selected to accept the proposed new basal delivery rate and a Reject Change button 1606 that rejects the change and keeps the basal delivery rate at the current rate. Once the patient makes a choice to accept or reject the new basal delivery rate, the chosen basal delivery rate is shown in section 1608 on the display of the management device 1601.

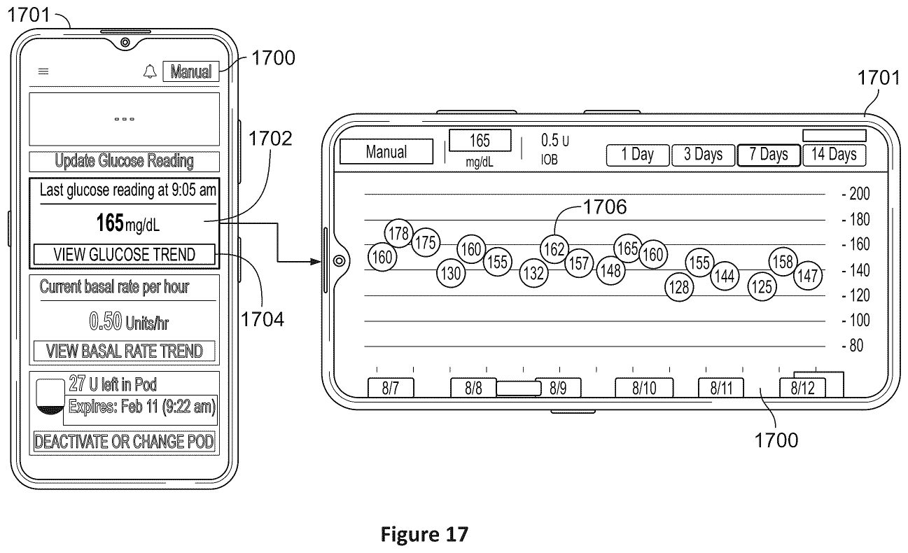

FIG. 17 depicts that the patient in exemplary embodiments may navigate to a plot of the glucose trend from the home screen while in manual mode. The home screen is shown on the display 1700 of the management device 1701. Section 1702 of the home screen includes a View Glucose Trend button 1704. The patient may select the View Glucose Trend button 1704 in order to have the 1706 of the manually entered glucose level readings over time displayed.

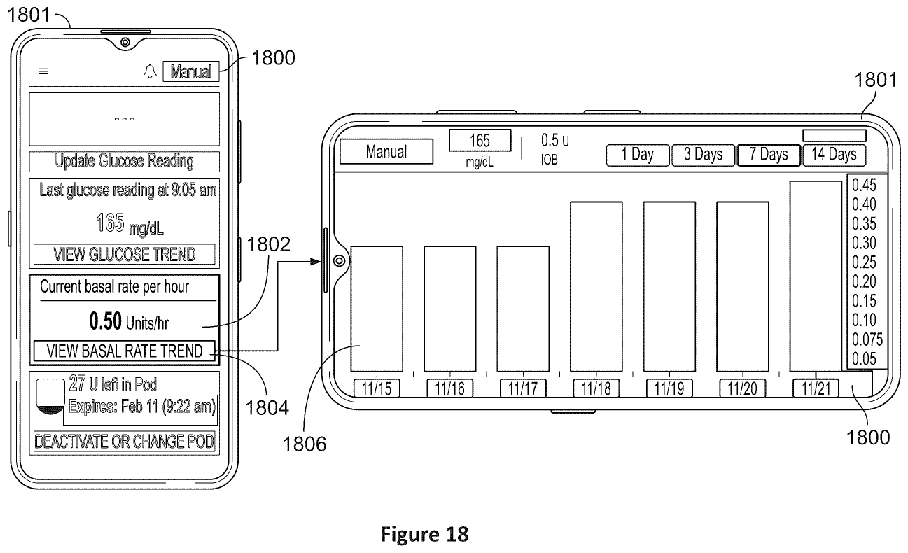

FIG. 18 depicts that the patient in exemplary embodiments may navigate from the home screen in manual mode to see the basal delivery rate trend. The home screen is displayed on display 1800 of the management device 1800. Section 1802 includes a View Basal Rate Trend button 1804 that may be selected by the patient to display a bar chart 1806 of the basal delivery rates over time on the display 1800 of the management device 1801.

It should be appreciated that the user interfaces discussed above are intended to be merely illustrative and not limiting. Other user interfaces may be displayed by the management device 104.

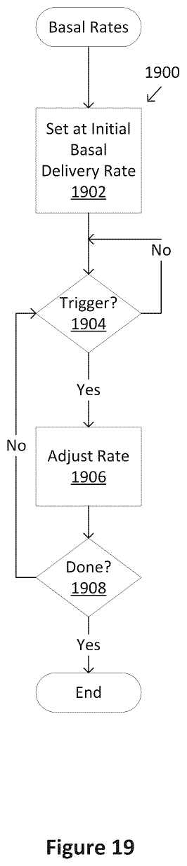

As has mentioned above, titration is an iterative process wherein adjustments in the basal delivery rate are made until the process settles on a basal delivery rate that provides good glucose level control for the patient. FIG. 19 depicts a flowchart 1900 of illustrative steps that may be performed in the titration process. At 1902, the basal delivery rate is set at an initial rate. As was discussed above, the initial rate may be 10 units per day, or lower, for all patients or the initial rate may be set per ADA guidelines based on weight. Other factors such as age and gender may also be taken into account in setting the initial rate. After the initial basal delivery rate is set, the system will lookout for triggers at 1904 that will cause an adjustment in the basal delivery rate at 1906. The triggers may take many forms, such as the elapsing of a time period, like a 6 hour time period. Other triggers may be blood glucose level events, like a hypoglycemic event or a hyperglycemic event. Other triggers may be the time of day, such as morning, evening arriving, and when a user goes to sleep. If the basal proves to be effective at providing good glucose control at 1908, the process may stop. Optimal glucose control may be determined by examining the magnitude of difference between the average glucose level for a time window and the target glucose level for the time window and seeing if the magnitude of difference is small enough to be acceptable. Other metrics instead may be applied in determining whether the glucose control is good and the titration process stopped.

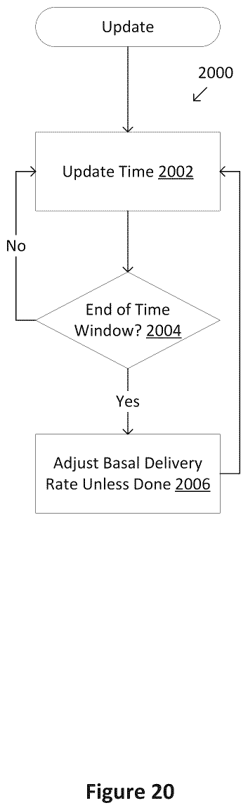

FIG. 20 depicts a flowchart 2000 of illustrative steps that may be performed in exemplary embodiments where the trigger is the elapsing of a time window. At 2002, the control application 116 of the medicament delivery device 102 may update the current time by receiving a clock input or other time reference. At 2004, a check is made to determine whether the end of the time window is reached. If the end of the time window is reached, at 2006, the basal delivery rate is adjusted unless the process is done as described above. Otherwise, the system waits and repeats the process beginning at 2002.

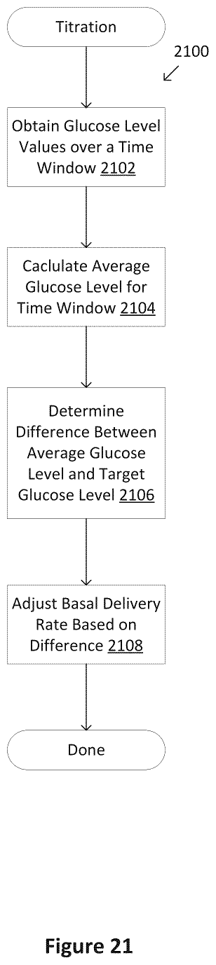

FIG. 21 depicts a flowchart 2100 of illustrative steps that may be performed in exemplary embodiments in performing the titration adjustment process. At 2102, the glucose level values for a time window are obtained. As was discussed above, the glucose level values may be obtained in automatic mode from a CGM or may be manually entered in manual mode. At 2104, the average glucose level of the patient for the time window is calculated from the obtained glucose level values. At 2106, the average glucose level is compared to a target glucose level value (e.g., 120 mg/dL) to determine the difference. At 2108, the basal delivery rate of the medicament delivery device is adjusted based on the difference. The adjusted basal delivery rate is the one used for the next time window of operation of the medicament delivery device.

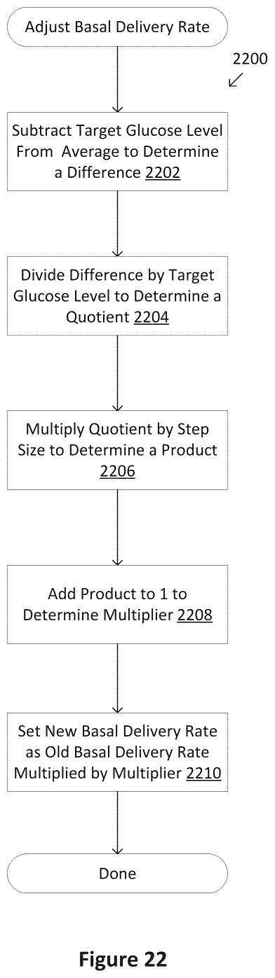

FIG. 22 depicts a flowchart of illustrative steps that may be performed in exemplary embodiments to adjust the basal delivery rate as part of the titration. Suitable formula for calculating the basal delivery rate where the medicament is insulin in exemplary embodiments is:

$${B}_{new} = B ( 1 + 0.2 * \frac{G - 120}{120} )$$

where B is the current basal delivery rate, G is the average glucose level for the time window, and B_{new} is the new basal delivery rate. Thus, at 2202, the target glucose level for the patient (e.g., 120 mg/dL) is subtracted from the average glucose level for the time window, G. At 2204, the difference is divided by the glucose level target for the patient to yield a quotient,

$$\frac{G - 120}{120} .$$

At 2206, the quotient is multiplied by a step size. In the example provided herein, the step size is 0.2. At 2208, the resulting product is added to 1 to produce a multiplier. The new basal delivery rate, B_{new}, is set as the old basal delivery rate, B, multiplied by the multiplier.

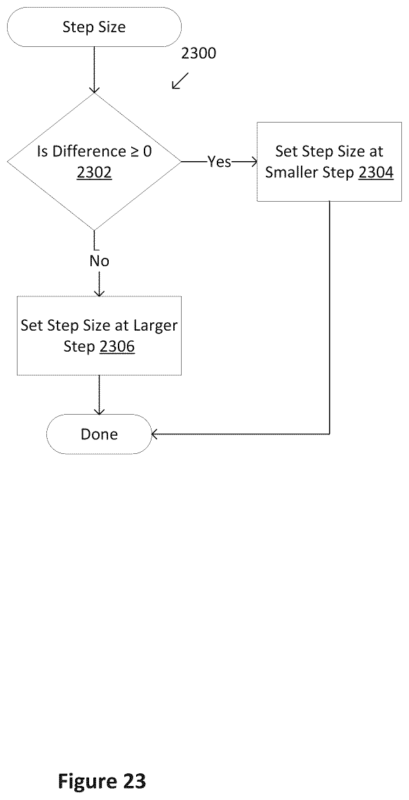

In some embodiments a same step size (e.g., 0.2) is used regardless of the difference between the average glucose level value of the patient and the target glucose level. In other embodiments, step size may vary depending on the difference between the average glucose level and the target glucose level. FIG. 23 depicts a flowchart 2300 of illustrative steps that may be taken in exemplary embodiments to use different step sizes. The thinking behind this approach is that the system should be more heavily biased towards avoiding hypoglycemia than avoiding hyperglycemia. Thus, positive adjustments in basal delivery rates may be more aggressive when the average glucose level of the patient is above target versus when the average glucose level of the patient is below target. At 2302, the difference between the average glucose level of the patient and the target glucose level is compared to zero to determine if the difference is negative. If the difference is negative at 2304, the step size is set at a smaller step size, like 0.2. On the other hand, at 2306, if the difference is not negative, the step size is set at a larger step size, like 0.4, to make adjustments more rapidly.

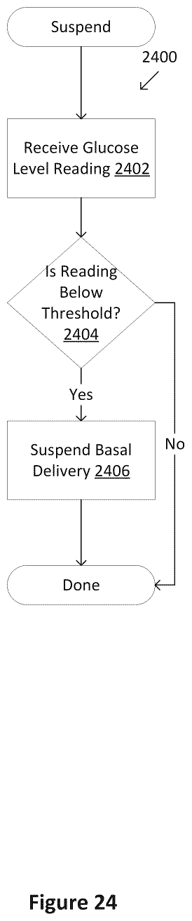

One possible trigger is if a glucose level reading for the patient is below the hypoglycemic threshold. FIG. 24 depicts a flowchart 2400 of illustrative steps that may be performed in exemplary embodiments to suspend basal delivery in some circumstances. At 2402, a glucose level reading for a patient is received. At 2404, a check is made whether the glucose level reading is below the hypoglycemic threshold. If the glucose level reading is below the hypoglycemic threshold, at 2406, the basal medicament delivery is suspended. The suspension may continue for a period of time or may continue until the glucose level of the patient rises to an acceptable level.

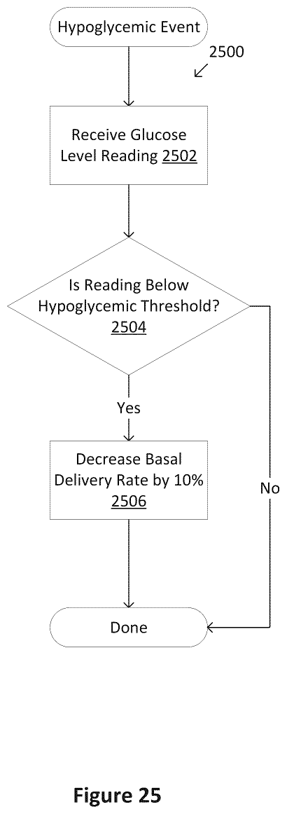

FIG. 25 depicts another option for responding to a low glucose level reading. FIG. 25 depicts a flowchart of illustrative steps that may be performed in exemplary embodiments to adjust the basal delivery rate when the glucose level of a patient falls below a hypoglycemic threshold. At 2502, a glucose level reading for the patient is received. At 2504, a check is made whether the glucose level reading is below the hypoglycemic threshold. At 2506, if the glucose level reading is below the hypoglycemic threshold, the basal delivery rate may be decreased by 10% or another suitable level of decrease.

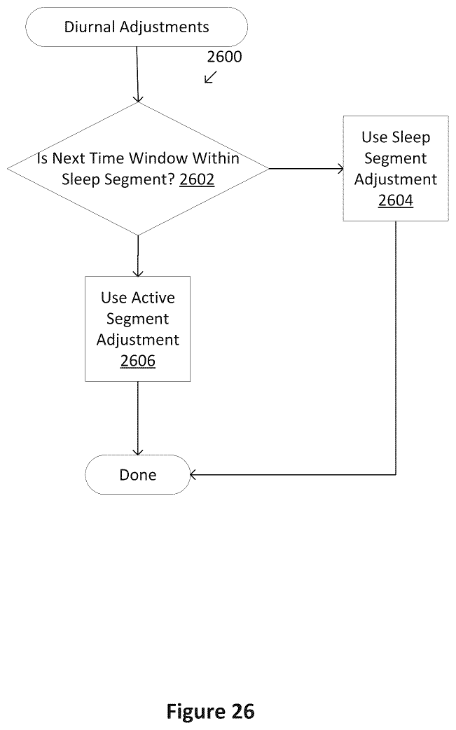

Adjustments in the basal delivery rate may also be based upon the time of day. FIG. 26 depicts a flowchart 2600 of illustrative steps that may be performed in exemplary embodiments to adjust the basal delivery rate based on time of day. In particular, the approach of FIG. 26 divides the day into a night sleep period when the patient sleeps and a daytime active period when the patient is awake. At 2602, when an adjustment is to be made, a check is made whether the next time window for which the adjustment is being made is within the sleep time segment for the patient. If so, at 2604, the sleep segment adjustment is used. Otherwise, the active segment adjustment is used.

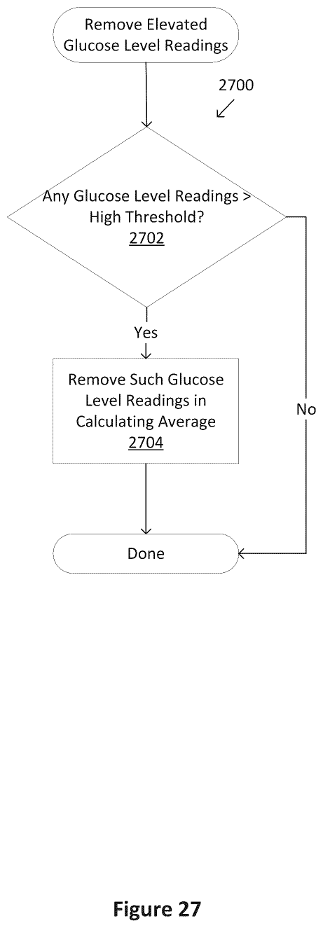

Meals can affect the basal delivery rate adjustments. Thus, it may be desirable to account for post-prandial elevated glucose levels in the titration process. FIG. 27 depicts a flowchart 2700 of illustrative steps that may be performed in exemplary embodiments to account for such elevated glucose levels. At 2702, a check is made whether any of the glucose level readings for a time window exceed a high threshold. If so, at 2704, those elevated glucose level readings are removed when calculating the average glucose level for the time window.

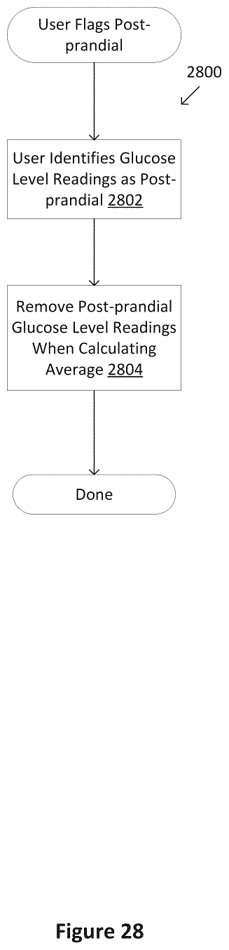

Another option is to have the patient help identify such post-prandial glucose level readings. FIG. 28 depicts a flowchart 2800 of illustrative steps that may be performed in exemplary embodiments in such a strategy. At 2802, the patient identifies glucose level readings for a time window as post-prandial glucose level readings, such as through a user interface on the management device. At 2804, the identified glucose level readings are removed when calculating the average glucose level for the time window.

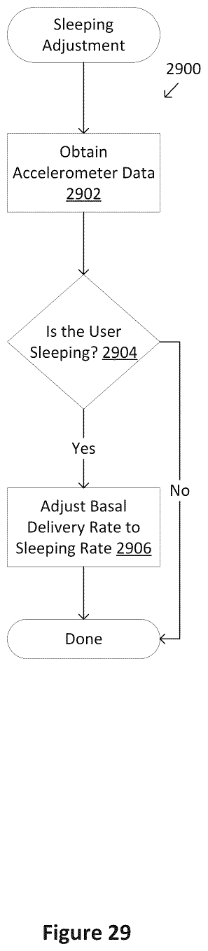

The basal delivery rate may be adjusted based on a detected activity of the user 108. FIG. 29 depicts a flowchart 2900 of steps that may be performed in exemplary embodiments to detect whether the user 108 is sleeping and to respond to detecting that the user 108 is sleeping. At 2902, data from the accelerometer 150 may be obtained. The data may indicate whether the user 108 is laying down and/or whether the user has largely been still for periods like when sleeping. Based at least in part on the accelerometer data, at 2904, a determination may be made of whether the user 108 is sleeping. The data represents the activity of the user 108 over a period. The obtained data may be compared to known patterns representative of the user sleeping. In some embodiments, other information, such as the time of day may be used in conjunction with the data to make the determination. If it is determined that the user 108 is sleeping, at 2906, the basal delivery rate may be adjusted for the user 108 sleeping. The adjustment may involve decreasing the basal delivery rate while sleeping. If the user 108 is determined to not be sleeping, no adjustment of the basal delivery rate is initiated.

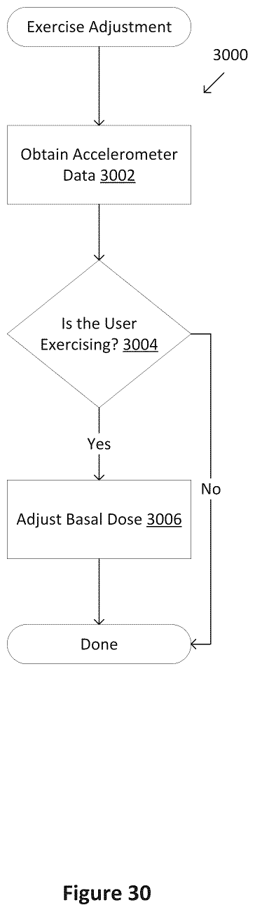

The basal delivery rate may be adjusted in response to the user 108 exercising. FIG. 30 depicts a flowchart 3000 of illustrative steps that may be performed in exemplary embodiments to detect whether the user 108 is exercising and to respond to such detecting. At 3002, data may be obtained from the accelerometer 150. Based on the data, at 3004, a determination may be made of whether the user 108 is exercising. The data may be compared to patterns of data for the user exercising to make the determination. If the user 108 is determined to not be exercising, no adjustment is initiated. However, if the user 108 is determined to be exercising, at 3006, the basal delivery rate may be adjusted. For instance, the basal delivery may be suspended or decreased while the user 108 is exercising. The adjustment may be undone once the user 108 completes exercising.

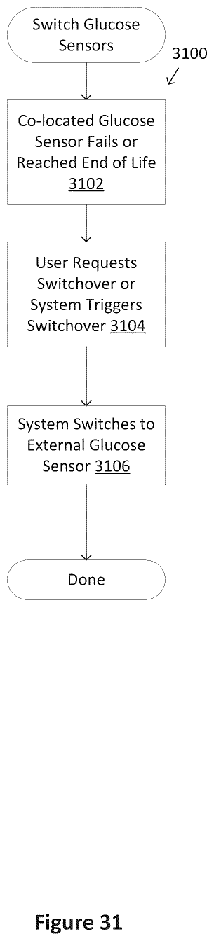

The medicament delivery device may rely upon the less accurate but cheaper glucose monitor that is one of the co-located integrated sensor(s) 106. Co-located is used herein to refer to the glucose sensor being integrated or packaged together, such as within a common housing or having another connecting or common structure. As mentioned above, in some exemplary embodiments, this glucose sensor may use microneedles. The exemplary embodiments may account for when such a glucose sensor malfunctions or reaches its end of life before the medicament delivery device 102 does. FIG. 31 depicts a flowchart 3100 of illustrative steps that may be performed by exemplary embodiments in such an instance. At 3102, the co-located glucose sensor fails, malfunctions, or reaches its end of life so as to not be operational. At 3104, the user 108 may detect the failure and request a switchover to an external glucose sensor. Alternately, the control application 116 or 120 may detect that the co-located integrated glucose sensor is no longer working and may trigger a switchover. At 3106, the control application 116 or 120 then switches to using the external glucose sensor, such as a CGM that is one of the external sensors 106. This allows the medicament delivery device to remain operational despite the co-located glucose sensor being non-operational.

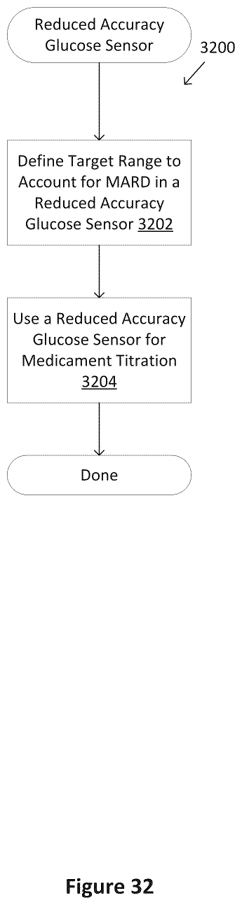

A reduced accuracy glucose sensor may be used for medicament titration with medicament delivery device 102 for treating Type 2 diabetes. The reduced accuracy glucose monitor may be cheaper to manufacture or purchase and may be more readily co-located with the medicament delivery device 102 than a higher accuracy CGM. A reduced accuracy glucose sensor may be one with a mean absolute relative difference (MARD) of greater than 10%. MARD is an accuracy estimate for glucose sensors that is widely accepted in the glucose sensor industry. MARD reflects the average relative difference between glucose level readings from a glucose monitor and glucose level readings obtained user finger sticks. FIG. 32 depicts a flowchart 3200 of illustrative steps that may be performed in exemplary embodiments with such a reduced accuracy glucose monitor. At 3202, a target range for glucose levels for the user 108 may be defined to account for the MARD of the glucose sensor. Thus, for example, if the standard target glucose level range for the user 108 is 70-180 mg/dL, the MARD adjusted target glucose level range may be 120-200 mg/dL to account for the reduced accuracy and to provide a bias to avoid hypoglycemia. At 3204, the reduced accuracy glucose monitor may be used to perform medicament titration for the user 108.

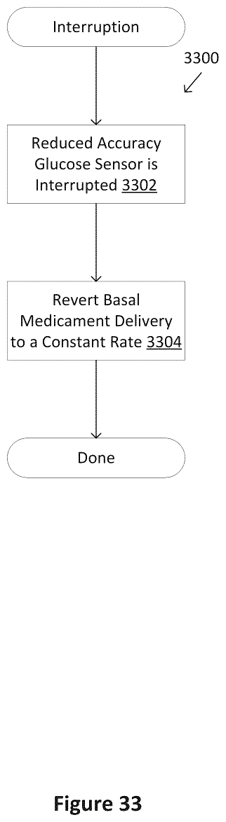

FIG. 33 depicts a flowchart 3300 of illustrative steps that may be performed in exemplary embodiments to address instances when such a reduced accuracy glucose monitor becomes interrupted for a period. At 3302, the reduced accuracy glucose sensor may be interrupted for a period so that the glucose sensor is not operational, the glucose sensor is not producing acceptable glucose level readings, or the glucose level readings are not be properly communicated to the medicament delivery device 102 or the management device 104. In such instances, at 3304, the medicament delivery rate by the medicament delivery device 102 may be set at a constant rate until the interruption ends. This allows the user 108 to continue to receive medicament at a safe and known rate despite the interruption.

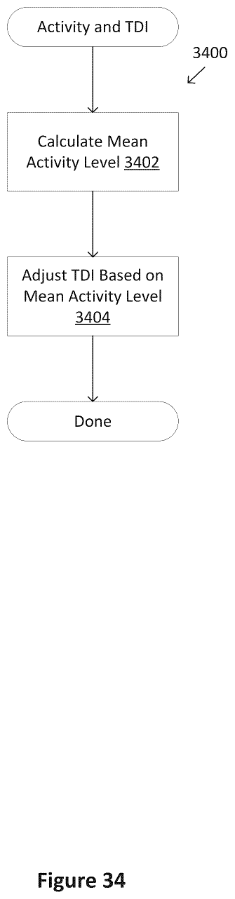

The accelerometer 150 may be used in adjusting the total daily insulin (TDI) value for the user 108. The adjusted TDI value may be used by the control application 116 or 120 in setting basal delivery doses. The accelerometer 150 may be used to measure activity levels, and the activity levels may be used to account for the change in TDI due to activity. FIG. 34 depicts a flowchart of illustrative steps that may be performed in exemplary embodiments to adjust the total daily insulin (TDI) for the 108 user based on activity level. At 3402, a mean activity level of the user 108 for a period may be calculated. At 3404, the TDI of the user 108 may be adjusted based on the mean activity level. For example, the TDI of a very active user 108 may be reduced due to being very active.

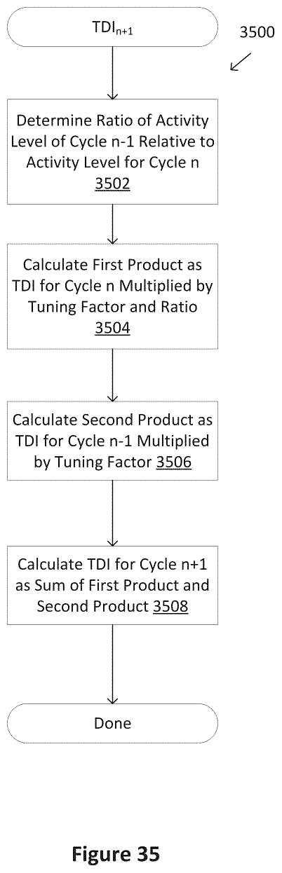

FIG. 35 depicts a flowchart 3500 of illustrative steps that may be performed in exemplary embodiments to adjust the TDI for the user 108 based on activity level per a first approach. The adjustment may be expressed by the following equation:

$${TDI}_{n + 1} = 0.2 \cdot {TDI}_{n} \frac{{( \overline{{A}_{x} + {A}_{y} + {A}_{z}} )}_{n - 1}}{{( \overline{{A}_{x} + {A}_{y} + {A}_{z}} )}_{n}} + 0.8 \cdot {TDI}_{n - 1}$$

where TDIₙ is the TDI value for cycle n, TDIₙ₋₁ is the TDI value for cycle n−1, TDIₙ₊₁ is the TDI value for cycle n+1, Aₓ, A_{y}, and A_{z} are the accelerometer values for a cycle in X, Y, and Z dimensions, respectively, (A̅ₓ̅+̅A̅_̅{̅y̅}̅+̅A̅_̅{̅z̅}̅)ₙ₋₁ is the sum of the magnitude of the accelerometer values for cycle n−1, and (A̅ₓ̅+̅A̅_̅{̅y̅}̅+̅A̅_̅{̅z̅}̅)ₙ is the sum of the magnitude of the accelerometer values for cycle n.

At 3502, a ratio of activity level of the user 108 during cycle n−1 to the activity level of the user 108 during cycle n is calculated. In other words,

$$\frac{{( \overline{{A}_{x} + {A}_{y} + {A}_{z}} )}_{n - 1}}{{( \overline{{A}_{x} + {A}_{y} + {A}_{z}} )}_{n}}$$

may be calculated. The cycles are operational cycles of the medicament delivery device, which may be a fixed duration in length, such as 5 minutes. The value n is a positive integer and constitutes the index of the cycle. For instance, where the cycles are 5 minutes in duration, there are 288 cycles per day, and n may assume a value between 1 and 288 and increase by an increment of 1 for each successive cycle. At 3504, a first product is calculated. The first product is calculated as the product of the ratio with the TDI for cycle n (i.e., TDIₙ) and an adjustable tuning factor (e.g., 0.2 in this instance). At 3506, the second product is calculated as the product of TDI for cycle n−1 (i.e., TDIₙ₋₁) with an adjustable tuning factor (e.g., 0.8). At 13508, the TDI for cycle n+1 (i.e., TDIₙ₊₁) is calculated as the sum of the first product with the second product. It should be appreciated that n need not be a cycle but can be another duration of time, such as a quarter of an hour, a half hour, an hour, a day, etc.

An alternative approach for adjusting is to adjust TDI for the user 108 based on what duration of time in a period is the user 108 presumptively exercising based on accelerometer data. A suitable equation for making such an adjustment is:

$${TDI}_{n + 1} = 0.2 \cdot {TDI}_{n} \frac{{Ex}_{n - 1}}{{Ex}_{n}} + 0.8 \cdot {TDI}_{n - 1}$$

$$where$$

$${Ex}_{n} = \frac{{T}_{n , {A}_{x} , {A}_{y} , {A}_{z} > th}}{{T}_{n}}$$

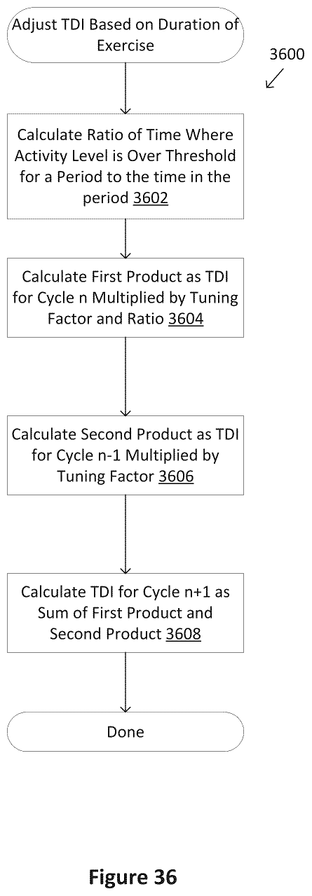

Exₙ is the ratio of time in a period n (e.g., a day) where the accelerometer data for the dimensions x, y, and z exceeds a threshold th, 0.2 and 0.8 are adjustable tuning parameters. FIG. 36 depicts a flowchart 3600 of illustrative steps that may be performed in exemplary embodiments to make the adjustment to the TDI of the user 108 based on the accelerometer data exceeding a threshold. At 3602, a ratio of time where the activity level is over a threshold (as measured by accelerometer data) during a period to the total time in the period n

$$( i . e . , \frac{{Ex}_{n - 1}}{{Ex}_{n}} )$$

may be calculated. At 3604, the first product may be calculated as the product of the TDI for the cycle n with an adjustable tuning factor and the ratio. At 3606, a second product may be calculated as the TDI for cycle n−1 multiplied by a tuning factor (e.g., 0.8). At 3608, the TDI for cycle n+1 is calculated as the sum of the first product with the second product.

While exemplary embodiments have been described herein, it should appreciated that various changes in form and detail may be made without departing from the appended claims.

1. A medicament delivery device configured for delivering basal medicament doses to a type two diabetes patient, comprising:
a non-transitory computer-readable storage medium storing computer programming instructions; and
a processor configured for executing the computer programming instructions, wherein executing the computer programming instructions causes the processor to:
establish an initial basal medicament delivery rate for a type two diabetes patient;
receive glucose level readings of the type two diabetes patient over an initial time window since establishing the initial basal medicament delivery rate;
as part of titration, adjusting the basal medicament delivery rate to new delivery rate for the type two diabetes patient based on the received glucose level readings; and
causing the delivery of basal medicament to the type two diabetes patient at the new delivery rate for a next time window.

2. The medicament delivery device of claim 1, wherein processor is further configured to receive input and establish the initial basal medicament delivery rate based at least in part on the received input.

3. The medicament delivery device of claim 2, wherein the input includes at least one of an age of the patient, a weight of the patient, a gender of the patient, a starting medicament dose, or a titration frequency.

4. The medicament delivery device of claim 3, wherein the input includes a titration frequency and the titration frequency determines a length of the initial time window and the next time window.

5. The medicament delivery device of claim 2, wherein the input is received wirelessly from a management device that manages the medicament delivery device.

6. The medicament delivery device of claim 5, wherein the management device is a smartphone.

7. The medicament delivery device of claim 1, wherein the processor is further configured to receive a request to pause medicament delivery and to pause delivery of medicament in response to receipt of the request.

8. The medicament delivery device of claim 1, wherein the processor is further configured to receive an indication that the patient will be in a state that will require less medicament during a portion of the next time window and is further configured to reduce the new medicament delivery rate during the portion.

9. The medicament delivery device of claim 1, wherein the processor is further configured to detect that the glucose level for the patient is below a threshold and to suspend delivery of medicament in response to the detecting.

10. The medicament delivery device of claim 1, wherein the processor is further configured to receive input indicating that the patient is about to sleep or about to begin an activity that will lower their glucose level and to adjust the new delivery rate to a lower delivery rate to compensate.

11. The medicament delivery device of claim 1, wherein the processor is further configured to adjust the new delivery rate based on time of day.

12. The medicament delivery device of claim 1, wherein the processor is further configured to remove an impact of meal consumption on the received glucose level readings in setting the new delivery rate.

13. The medicament delivery device of claim 1, wherein the medicament is insulin, a glucagon-like peptide-1 (GLP-1) receptor agonist, a glucose-dependent insulinotropic polypeptide (GIP) receptor agonist, a dual GLP-1 and GIP receptor agonist, an antihyperglycemic, or a co-formulation thereof.

14. A management device for an medicament delivery device, said management device comprising:
a non-transitory computer-readable storage medium storing computer programming instructions;
a display; and
a processor configured for executing the computer programming instructions, wherein executing the computer programming instructions causes the processor to:
display on the display a user interface to obtain input regarding gender, age and weight of the patient;
obtain the input regarding gender, age and weight of the patient; and
forward the input regarding gender, age and weight of the user to the medicament delivery device for the patient to initiate titration of a medicament delivery rate to the patient.

15. The management device of claim 14, wherein the obtained input also includes an identification of a starting medicament dose at which the titration is to begin and an indication of how often to titrate.

16. The management device of claim 14, wherein the processor is further configured to display another user interface to receive an indication that the patient is about to sleep or is about to exercise and the processor is further configured to forward the received indication to the medicament delivery device.

17. The management device of claim 14, wherein the medicament is insulin, a glucagon-like peptide-1 (GLP-1) receptor agonist, a glucose-dependent insulinotropic polypeptide (GIP) receptor agonist, a dual GLP-1 and GIP receptor agonist, an antihyperglycemic, or a co-formulation thereof.

18. A medicament delivery device configured for delivering basal medicament doses to a type two diabetes patient, comprising:
a non-transitory computer-readable storage medium storing computer programming instructions; and
a processor configured for executing the computer programming instructions, wherein executing the computer programming instructions causes the processor to:
establish an initial basal delivery rate of medicament for delivery to the patient by the medicament delivery device over an initial time window;
increase the basal delivery rate in subsequent time windows based on glucose level readings for the patient;
decrease the basal delivery rate to a decreased basal rate for a time window after the time windows where the basal delivery rate is increased; and
where the glucose level readings of the patient for the later time window exhibit good glucose level control, conclude the titration by setting the decreased basal rate as the basal delivery rate for the patient.

19. The medicament delivery device of claim 18, wherein executing the computer programming instructions causes the processor to calculate average glucose levels for glucose readings of the time windows.

20. The medicament delivery device of claim 19, wherein executing the computer programming instructions causes the processor, for the time windows, to calculate differences between the average glucose levels of the time windows and target glucose levels.

21. The medicament delivery device of claim 20, wherein the increases of the basal delivery rate are based on the calculated differences.

22. The medicament delivery device of claim 21, wherein the device is an insulin pump.

23. The medicament delivery device of claim 18, wherein the medicament is insulin, a glucagon-like peptide-1 (GLP-1) receptor agonist, a glucose-dependent insulinotropic polypeptide (GIP) receptor agonist, a dual GLP-1 and GIP receptor agonist, an antihyperglycemic, or a co-formulation thereof.
